(19) United States
(12) Patent Application Publication
(10) Pub. No.: US 2006/0063979 A1
(43) Pub. Date: Mar. 23, 2006

(54) Retracting devices

(76) Inventors: Kenneth Rosenblood, Los Angeles, CA (US); Christopher N. Quan, Quincy, MA (US); William Dorfman, Beverly Hills, CA (US)

Correspondence Address: Michael Bergman;Bergman Kuta LLP, P.O. Box 400167, Cambridge, MA, 02140, US

(21) Appl. No.: 11/203,540

(22) Filed: Aug. 12, 2005

Related U.S. Application Data
(63) Continuation-in-part of application No. 11/173,297, filed on Jun. 30, 2005.
Application No. 11/173,297 is a continuation-in-part of application No. 11/173,839, filed on Jun. 30, 2005.
(60) Provisional application No. 60/604,577, filed on Aug. 25, 2004.
(60) Provisional application No. 60/697,019, filed on Jul. 5, 2005.

(51) Int. Cl. A61B 1/32

(52) U.S. Cl. 600/237

(57) ABSTRACT

The present invention relates to a retracting device for retracting at least a portion of a user's mouth. The retracting device includes formations, which may be inter-engaging and/or non-inter-engaging with other dental tools or apparatus. The formations are adapted for repeatably positioning a subject's mouth with respect to a light system, and/or an imaging film, and/or a dental tray.

### CROSS-REFERENCE TO RELATED APPLICATION

The present application claims the benefit of U.S. provisional application Nos. 60/604,577, filed Aug. 25, 2004, entitled “Lip Retractors”; and 60/697,019, filed Jul. 5, 2005, entitled “Framework Retracting Devices”, the entire contents of which are hereby expressly incorporated by reference.

The present application is a continuation-in-part of U.S. patent application Ser. No. 11/173,297, filed Jun. 30, 2005, entitled “Retracting Devices”; and Ser. No. 11/173,839, filed Jun. 30, 2005, entitled “Illumination Systems for Dentistry Applications”; the contents of which are hereby expressly incorporated by reference.

The present application includes claims that may be related to the claims of U.S. patent application Ser. No. 11/173,274, filed Jun. 30, 2005, entitled “Dental Light Devices Having an Improved Heat Sink”; Ser. No. 10/______, filed Jun. 30, 2005, entitled “Voice Alert System for Dentistry Applications”; Ser. No. 10/______, filed Jun. 30, 2005, entitled “Light Guides for Dentistry Applications”; Ser. No. 10/______, filed Jun. 30, 2005, entitled “Support Systems for Dentistry”; Ser. No. 11/173,076, filed Jun. 30, 2005, entitled “Curing Light Capable of Multiple Wavelengths”; Ser. No. 11/173,264, filed Jun. 30, 2005, entitled “Curing Light”; the contents of all of which are hereby incorporated by reference.

### FIELD OF THE INVENTION

This invention relates to Retracting devices for retracting the lips in general. Specifically, this invention relates to retracting devices suitable for use in dental examination and/or treatment.

### BACKGROUND OF THE INVENTION

Mouth corner spreading devices, also known as cheek retractors or tongur cups, are well known in the art for spreading portions of the lips, which spread the cheeks, for examination and/or treatment by healthcare professionals. Exemplary mouth corner spreading devices include devices that spread a portion or several portions of the upper and lower lips using levers that are biased apart by an assistant, using flanges that cup and spread the lips, using devices that include metal resilient members, and using devices that have two retaining members for spreading two portions of the lips. However, there is still a need for a retractor as described below for the advantages that are associated therewith.

### SUMMARY OF THE INVENTION

According to the present invention, there is provided a disposable retracting device for retracting at least a portion of a user's lips. The device may be adapted for a one-patient use or a one-use, whenever appropriate.

According to a first embodiment of the invention, the retracting device includes at least two channel retainers or flanges and at least two resilient members, wherein each channel retainer includes a framework having a suitable elastomeric material disposed within the framework, the elastomeric material being adapted to provide more comfort for the patient and also to better accommodate different lip sizes. The elastomeric material may be amenable to limited sterilization or autoclaving. In one aspect, the elastomeric material is adapted to hold at least a portion of a subject's lips in its fold.

In one embodiment, each resilient member may be integrally molded to two adjacent channel retainer frames. The resilient member may include an arch. In another aspect, the elastomeric material is conformable and may be adjustable to fit different sizes of lips comfortably, as noted above. In a further aspect, the resilient members may be made of wire-like materials or materials with substantially rounded cross-sections adapted to provide more comfort by minimizing sharp edges. In yet another aspect, at least one resilient member includes at least two separate portions, one of the portion includes at least one formation for inter-engaging with a corresponding formation of the second portion when the two portions are apposed. The two portions may be lengthened or shortened to vary the size of the retracting device. The engagement may be substantially reversible or irreversible.

In another embodiment, at least one pair of pads may be disposed onto at least two adjacent resilient members. In one aspect, the pads may be integrally formed onto the resilient members. In anther aspect, the pads may be attached to the resilient members by an adhesive or heat sealing. In a further aspect, the pads may be amenable to limited sterilization or autoclaving.

In a second embodiment of the invention, a retracting device for retracting at least a portion of a user's lips includes at least two channel retainers, and a tongue retainer, wherein said at least two channel retainers being held in a spaced apart relationship by at least two resilient members, and the tongue retainer being attached to at least two of the channel retainers by two support members. In one aspect, the support members may be the same as the resilient members. In another aspect, the support members may be additional, secondary resilient members. In one embodiment, each of the channel retainers includes a framework having a suitable elastomeric material disposed within the framework, the elastomeric material being adapted to provide more comfort for the patient and also to better accommodate different lip sizes, In one aspect, the elastomeric material is adapted to hold at least a portion of a subject's lips in its fold. In another embodiment, the tongue retainer includes a framework made of a relatively high structural integrity with a suitable elastomeric material disposed within for holding the tongue comfortably, and the channel retainers do not include a framework. In a further embodiment, the tongue retainer and channel retainers include a framework having an elastomeric material disposed therein.

In one aspect, at least one resilient member includes at least two separate portions, one of which includes at least one formation for inter-engaging with a corresponding formation of the second portion when the two portions are apposed. The engagement may be substantially reversible or irreversible.

In a third embodiment, a lip retracting device includes four channel retainers, at least four resilient members, and an adjustable mechanism present on at least one resilient member such that the resilient member may be made to be adjustable to fit different mouth sizes.

In one aspect, at least one resilient member includes at least two separate portions, one of which includes at least one formation for inter-engaging with a corresponding formation of the second portion when the two portions are apposed. The engagement may be substantially irreversible.

In one embodiment, the channel retainers may include a framework having an elastomeric material disposed therein. In another embodiment, the retracting device includes a tongue retainer. In one aspect, the tongue retainer includes a framework having an elastomeric material disposed therein. In another aspect, the tongue retainer does not include a framework. In another embodiment, the channel retainers do not include a framework and the tongue retainer includes a framework having an elastoemric material disposed therein. In a further embodiment, the adjustable mechanism includes a one-way locking mechanism

In one aspect, the resilient members may have a sufficiently high elasticity such that their tendency to spring back to their expanded shape provides a biasing action for retracting at least a portion of the mouth or lip. In another aspect, the framework may have a relatively high structural integrity.

The present invention further includes a retracting device including a framework with an elastomeric material disposed therein, having formations, which may be inter-engaging and/or non-inter-engaging with other dental tools or apparatuses. The formations are adapted for repeatably positioning at least a portion of a subject's mouth with respect to a light system, and/or an imaging film, and/or a dental tray, and/or an apparatus adapted for aspiration, such as an aspirator, and/or a suction tube.

In one aspect, a light system includes a spacer such as a light output port, an imaging device, a light guide or an examination device including inter-engaging formations for removably inter-engaging the retracting device as the spacer and the retracting device become apposed. In one embodiment, at least one formation includes at least one wing-like member.

In another aspect, a retracting device includes formations such as a dental tray adapted for repeatably positioning a subject's teeth with a treatment composition.

In a further aspect, a retracting device includes formations such as an imaging device adapted for repeatably positioning a subject's teeth with respect to an imaging device, and/or a light source or imaging source.

In one embodiment, at least one resilient member may be attached to the frame work on the inside side wall of the two adjacent channel retainers by means of an adhesive or heat sealing. In one aspect, each of the resilient members may be integrally molded to the frame work of the inside side wall of the two adjacent channel retainers and includes two arches. In another aspect, each of the wing-like flanges or members is integrally molded to a channel retainer, spaced away from the attachment of a resilient member. In a further aspect, the wing-like member may be attached to a channel retainer by an adhesive or heat sealing. Each of the wing-like members may be adapted to fit into a slot in an output port, a light guide, an imaging device or an examination device such as a cone.

In another embodiment, a tongue retainer may be included.

Other alternatives and embodiments for practicing the invention are also described herein and further discussed below in the Detailed Description section.

### BRIEF DESCRIPTION OF THE DRAWINGS

These and other features and advantages of the present invention will become appreciated as the same become better understood with reference to the specification, claims and appended drawings wherein:

FIG. 1 depicts a semi-schematic perspective view of a retracting device provided in accordance to one practice of the present invention;

FIG. 1a depicts the retracting device of FIG. 1 when inserted into a user's mouth;

FIG. 1b depicts the retracting device of FIG. 1 including the suitable material;

FIG. 2 depicts a semi-schematic perspective view of a retracting device with a tongue retainer;

FIG. 2a depicts the retracting device of FIG. 2 when inserted into a user's mouth;

FIG. 3 depicts a semi-schematic perspective view of a retracting device with interface features for coupling to a dental light source;

FIG. 3a shows an exploded view of the combination of a lip retracting device with the light guide and a lamp;

FIG. 3b shows in perspective view, an illumination frame having slots according to one embodiment of the invention;

FIG. 3c shows, in perspective view, an illumination frame according to one embodiment of the invention;

FIG. 4 depicts a semi-schematic perspective view of another embodiment of a retracting device;

FIG. 4a depicts the retracting device of FIG. 4 in the ready-to-use mode;

FIG. 4b shows the retracting device of FIG. 4 including the suitable material;

FIG. 5 depicts a semi-schematic perspective view of a retracting device with a tongue retainer;

FIG. 6 depicts a semi-schematic perspective view of a retracting device with a tongue retainer and interface features for coupling to a dental light source;

FIG. 7 depicts a semi-schematic perspective view of a retracting device having pads attached;

FIG. 8 depicts a semi-schematic perspective view of a pad for attachment to a retracting device;

FIG. 9 depicts a semi-schematic perspective view of a retracting device with an attached dental tray;

FIG. 10 depicts a semi-schematic perspective view of a retracting device with an attached dental tray and interface features for coupling to a dental light source;

FIG. 11 depicts a semi-schematic perspective view of a retracting device having an adjustable mechanism;

FIG. 11a depicts a semi-schematic perspective view of a retracting device having a one-way adjustable locking mechanism;

FIG. 11b shows a semi-schematic perspective view of another embodiment of a retracting device having an adjustable mechanism;

FIG. 11c depicts a semi-schematic perspective view of another embodiment of a retracting device having an adjustable mechanism;

FIG. 11d shows a semi-schematic perspective view of an embodiment of a locking mechanism;

FIG. 11e depicts a semi-schematic perspective view of an embodiment of a locking mechanism;

FIG. 11f depicts a perspective view of a post;

FIG. 12 depicts a semi-schematic perspective view of a retracting device having a formation for holding an aspirator;

FIG. 13 depicts a semi-schematic perspective view of a retracting device having a formation for holding X-ray film or sensor;

FIG. 14 shows, in perspective view, a retracting device with extended wings according to an embodiment of the invention;

FIG. 15 shows, in perspective view, a retracting device with extended wings and targets according to an embodiment of the invention; and

FIG. 16 shows, in perspective view, a retracting device including a film holder according to an embodiment of the invention.

### DETAILED DESCRIPTION

The detailed description set forth below is intended as a description of the presently preferred device provided in accordance with aspects of the present invention and is not intended to represent the only forms in which the present invention may be practiced or utilized. It is to be understood, however, that the same or equivalent objectives and functions may be accomplished by different embodiments and components that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The retracting device includes inter-engaging and/or non inter-engaging formations. Inter-engaging formations include those formations that engage a device, an apparatus or tool with at least one corresponding formation in another device, apparatus or tool. Non-inter-engaging formations include those formations that bring such device, apparatus or tool into close proximity with at least a portion of a subject's mouth.

The word formation as used herein in relation to a retracting device and/or a dental system such as a light system, a light guide, an imaging system, a dental treatment composition, an imaging system, a spacer, a support system, or a dental tool or apparatus adapted for aspiration, such as an aspirator or a suction tube, refers to the portion of the dental system which is adapted to inter-fit with a corresponding portion of an adjoining dental system, or a component of a subject's mouth. A formation thus includes at least a portion of any of the above listed articles and may be formed or shaped by molding, or a formation may be formed separately.

Suitable inter-engaging formations include tongues and grooves, posts and sockets, swingable hooks and sockets, resilient clips and sockets, clips and protrusions or depressions, tongues or wing-like members and slots, balls and cavities, balls and sockets, some of which are more specifically exemplified in detail below. Non-inter-engaging formations include dental trays, imaging film holders, and other features adapted to position any dental treatment or imaging material in a patient's mouth.

Retracting devices may be made to be disposable for hygienic reasons. They may also be made to be more conformable for comfort and fit. One example of such devices may include devices having limited steriliability or autoclavabiity through the use of suitable materials, such as an elastomeric or foam materials, which can be more conformable and/or elastic, for better fit and comfort during use. In another example, the device may be manufactured in one-size having adjustable formations through the use of an adjustable locking mechanism for better fit. Other examples of disposable devices or limited use devices are also contemplated.

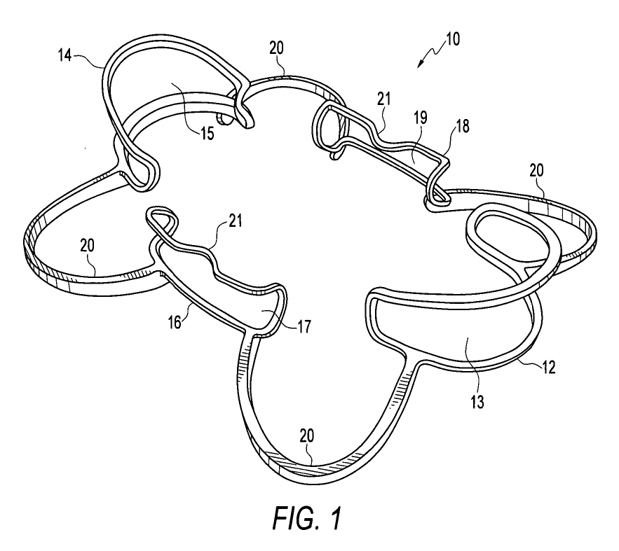

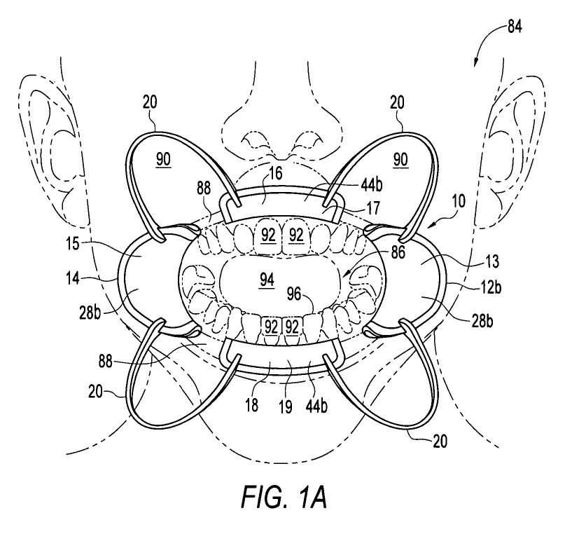

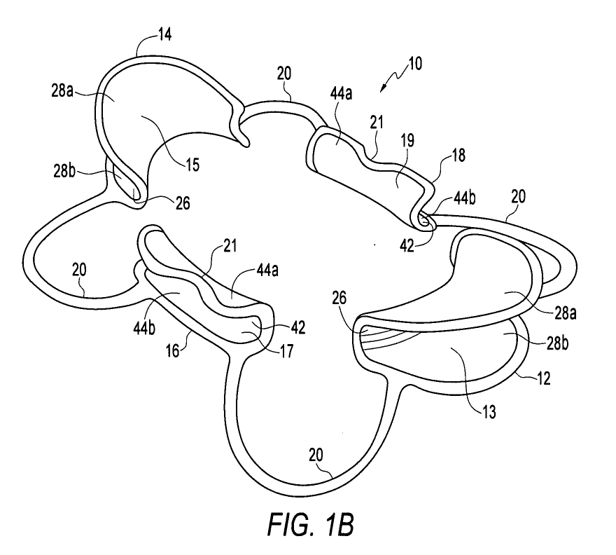

Referring now to FIG. 1, there is shown a retracting device for retracting the upper and lower lips (herein “lips”) for facilitating examination, imaging of the mouth and/or teeth, and/or the whitening or curing process in accordance to one practice of the present invention, which is generally designated 10. The retracting device 10, also known as a tongur cup, may generally include a framework adapted to define four spaced apart channel retainers, 12, 14, 16, 18, also known as flanges, for retaining four corresponding portions of the lips for examination and/or treatment of the mouth or teeth. The framework defines the boundary and general shape of each of the channel retainers, 12, 14, 16, 18, and an elastomeric material contained within the framework thereof, forming the bounded areas, 13, 15, 17, 19, respectively. Within the bounded areas, 13, 15, 17, 19, a suitable material may be mounted so as to form generally “U” shaped channels within the channel retainer frameworks, 12, 14, 16, 18. During use, the retracting device 10 draws back the lips, and retracts the cheeks, to expose the mouth so that a health care professional may more easily see the teeth and/or the inside of the mouth.

The four channel retainers include two side channel retainers, 12, 14 for retaining the ends of the lips, approximately where the upper and the lower lips intersect, and two lip channel retainers, 16, 18, for retaining the mid-section of the upper and lower lips. More particularly, the four channel retainers or flanges, 12, 14, 16, 18, may be adapted to cup the lips and bias them open to expose the teeth for treatment and/or examination.

The elastomeric material mounted within the framework of the channel retainers, 12, 14, 16, 18, defining the bounded areas, 13, 15, 17, 19, respectively, may be any material that provides comfort for a user/patient. For example, the elastomeric material, may be of sufficient softness to provide additional cushioning and of sufficient elasticity to allow it to follow the contour of a patient's lips. In one embodiment, the elastomeric material may be mounted in a slightly stretch form so that it is held taut against the framework. In another embodiment, the elastomeric material is held loosely within the framework.

Suitable materials may include any rubber or elastic foam. The rubber may be natural or synthetic. Synthetic rubbers may be, for example, elastomeric materials and may include, but not limited to, various copolymers or block copolymers(Kratons®) available from Kraton Polymers such as styrene-butadiene rubber or styrene isoprene rubber; EPDM (ethylene propylene diene monomer) rubber; nitrile (acrylonitrile butadiene) rubber; latex rubber; silicone rubber (polysiloxanes); Neoprene (polychloroprene) rubber and the like. Foam materials may be closed cell foams or open cell foams, and may include, but is not limited to, a polyolefin foam such as a polyethylene foam, a polypropylene foam, and a polybutylene foam; a polystyrene foam; a polyurethane foam; any elastomeric foam made from any elastomeric or rubber material mentioned above; or any biodegradable or biocompostable polyesters such as a polylactic acid resin (comprising L-lactic acid and D-lactic acid) and polyglycolic acid(PGA); polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers; and polyester/urethane resin.

A plurality of resilient members 20 may be incorporated in the retracting device 10 to interconnect the four channel retainers, 12, 14, 16, 18, together so that they may function as biasing means. When in use, as shown in FIG. 1a, the resilient members 20 are arched outwardly with respect to the center portion of the retracting device 10. The four channel retainers, 12, 14, 16, 18 cup respective portions of the lips, while the resilient members 20 provide a retractive force to retract the lips radially outwardly for examination and/or treatment.

The side channel retainers, 12, 14, also having an elastomeric material mounted within the bounded areas, 13, 15, resemble curvilinear c-channels, with the mounted material forming a flexible or conformable arcuate race 26 and two channel side walls 28a and 28b, as shown in FIG. 1b. As noted above, in one embodiment, The channel side walls resemble a bell shape and may include a maximum wall dimension at approximately the mid-point and two smaller tapered tips at the ends thereof.

The side channel retainers, 12, 14, when mounted with an appropriate material in bounded areas, 13, 15, further include an interior surface or inside side wall 28b and an exterior surface or outside side wall 28a. In one embodiment, the inside side wall or surface 28b, which is intraoral as further discussed below, may be slightly larger relative to the outside side wall. However, the relative dimensions can be reversed or can be the same without deviating from the functionality of the lip retracting device 10.

The arcuate race 26 may include a radius of curvature adapted to mimic the curvature of the side of the lips when the lips are in the opened position. Because this curvature may vary depending on the size and age of the user or patient, the retracting device 10 may be implemented with varying radii of curvature to fit the varied shape of the particular user/patient. The arcuate race 26 may also include an irregular curvature of two or more different radii of curvature. If implemented, the irregular curvature may vary the amount of retraction of the portion of the lip that is seated within the arcuate race to vary the amount of retraction between those portions of the lip. The two lip channel retainers, 16, 18, including an elastomeric material mounted therein in the bounded areas, 17, 19 may also have different radii of curvature, similar to the side channel retainers 12, 14.

The lip channel retainers, 16, 18, like the side channel retainers, 12, 14, resemble a curvilinear c-channel when mounted with elastomeric material therein in the bounded areas, 17, 19, in that they may include an arcuate race 42 and two channel side walls or surfaces 44a and 44b. In one embodiment, the elastomeric material may be mounted in a slightly stretch form so that it is held taut against the framework. In another embodiment, the elastomeric material is held in non-stretch form or loosely within the framework. The radius of curvature of the lip channel retainers may be larger than the radius of curvature of the side channel retainers, 12, 14. The larger radius of curvature may enable the lip channel retainers, 16, 18, to conform to the contour of the upper and lower lips near the frenum, which is more planar relative to the side of the lips. Depending on the size and age of the intended user/patient, the radius of curvature of the lip channel retainer, 16, 18, may also vary.

The elastomeric material may also provide better conformity and flexibility than a non-elastomeric material within the bounded area.

As shown, a frenum release 21 may be incorporated in the inside side walls 44b of the lip channel retainers, 16, 18, for providing relief to the frenum of the upper and lower lips. In one embodiment, the frenum release 21 may include a partial oval shaped cutout having a size sufficient to provide clearance for the frenum. In other words, the frenum release 21 may be such that the lowest most portion of the frenum release only slightly touches the frenum when in use, for example, and more for example, not touch the frenum at all. Although the oval shaped cutout is shown for the frenum release 21, a partial circle, a rectangular cutout, a square cutout, or other geometrical shaped cutout may also be incorporated without deviating from the function of the frenum release. The cut is present only on the framework.

The retracting device 10 may be made by injection molding or casting a thermoplastic material such as polypropylene, polyethylene, polystyrene, polyester, polycarbonate or the like. It may also be made out of biocompostable or biodegradable polymers, including those disclosed above. More for example, the lip retracting device may be made by injection molding polypropylene and may be a smooth and transparent finish. In another embodiment, the device may be opaque and colored, including white color.

The retracting device 10 may be made as a single piece or as separate components that may be subsequently assembled. The retracting device may also, owing to its framework structure, be formed by cutting a shape from a flat sheet of a suitable material, such as a thermoplastic mentioned above. The proper three-dimensional orientations for the various parts of the framework may be accomplished by heating and bending the plastic framework to achieve the desired shape. The shape may then be retained by cooling the framework.

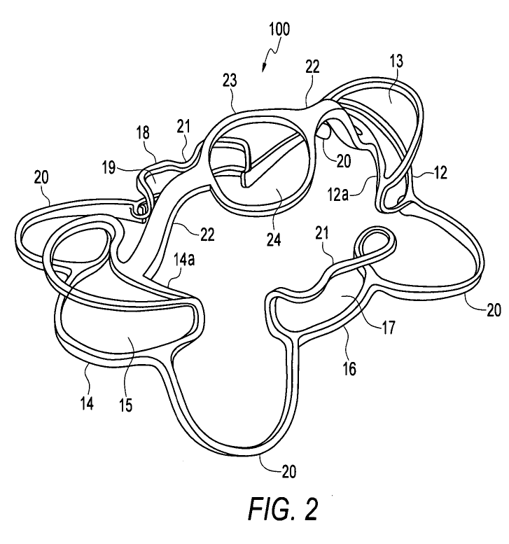

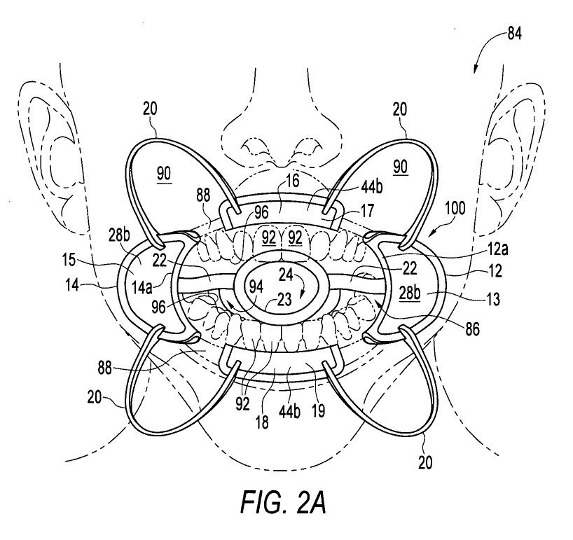

FIG. 2 shows a perspective view of another embodiment of a retracting device of the present invention. The retracting device 100 is substantially identical to the retracting device 10 of FIG. 1, with the only substantial difference being the addition of framework sections, 12a, 14a, forming arcuate races in the channel retainers, 12, 14, respectively, and the addition of a support or resilient members 22 that include a tongue retaining framework 23 which defines the bounded area 24. The arcuate framework sections, 12a, 14a, also divide the bounded areas, 15, 17, respectively and provide attachment support at their approximate midpoints for the support members 22. The bounded area 24 may be mounted with an appropriate material, such as the elastomeric material mentioned above, so as to form a cup for retaining the tongue in the back of the mouth when the retracting device 100 is in use on a patient, as shown in FIG. 2a. The elastomeric material may be of sufficient softness to provide additional cushioning and of sufficient elasticity to allow it to follow the contour of a patient's lips to provide more comfort during use on a patient. Suitable materials may include any of the elastomers mentioned above. When incorporated, the tongue retainer 23 and the support members 22 cooperate to block the tongue and limit the tongue to the back vicinity of the mouth, thus enabling access to the lingual portion or back of the teeth for examination and/or treatment. In short, the tongue retainer may be configured to minimize interference by the tongue during treatment and/or examination by a health care professional, as noted above.

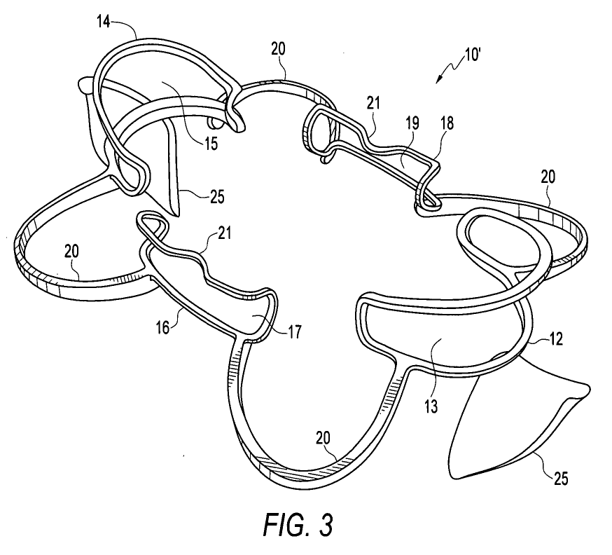

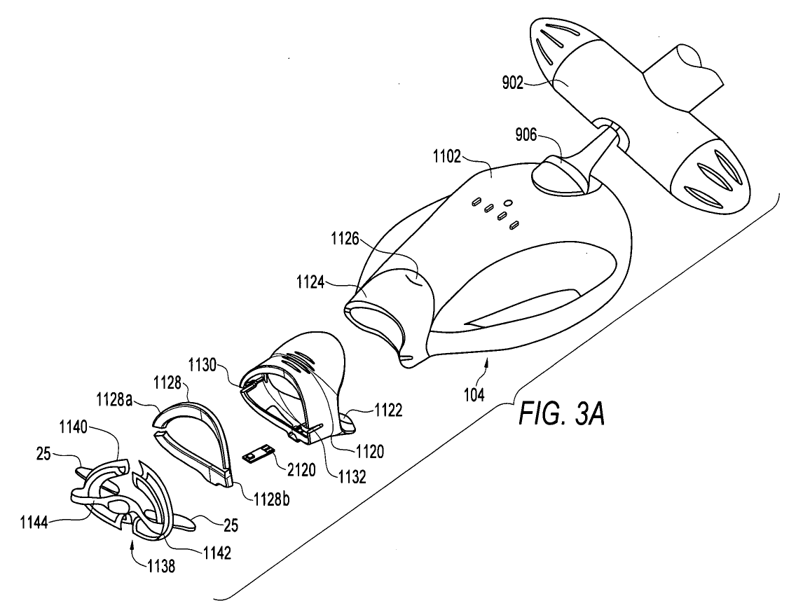

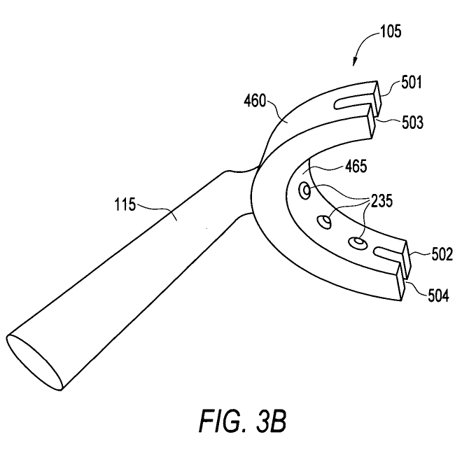

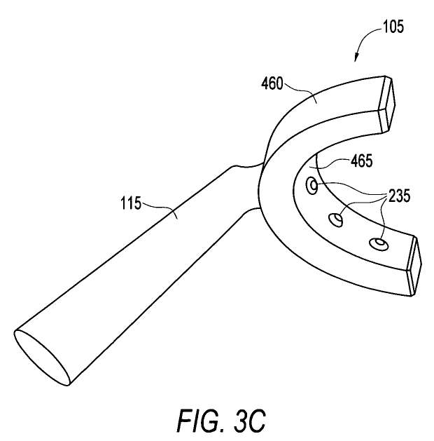

FIG. 3 shows a perspective view of another embodiment of a retracting device of the current invention. The retracting device 10′ may be substantially identical to the retracting device 10 of FIG. 1, with the only substantial difference being the addition of wing-like members 25, for example, which may be integrally molded or attached by an adhesive or heat sealing, to the framework of lip channel retainers, 12, 14. The wing-like members may be constructed of the same or different material as the frameworks of the channel flanges or retainers, or other parts of the retracting device, including the materials mentioned above, or of a more sturdy polymeric material or composite. Additionally, they may also be opaque or colored even if the rest of the lip retracting device may be colorless or clear.

In one embodiment, the wing-like flanges 25 may be designed for fitting into a pair of formations, such as slots 1130, 1132 formed in the output port, an imaging device, a lamp system, or a light guide 1120 of a lamp system 1102 used in a whitening process or to the slots in any examining device, such as that shown in an exemplary illumination system of FIG. 3a, an exploded view of a combination of a lip retracting device 1138, a light guide 1120 and a lamp system 1102. Another exemplary illumination or lamp system and the use of which is disclosed in Ser. No. 10/715,681, filed Nov. 17, 2003, which is expressly incorporated herein by reference as if set forth in full. The light source or sources, for example, may include a lamp, an arc lamp such as a halogen light source, semiconductor light emitting devices, light-emitting chips such as an LED, a solid state LED, an LED array, a fluorescent bulb, and so on. In the case of dental imaging, the light source may include the above in addition to a laser, an x-ray or even an infrared source. In another embodiment, instead of a light guide 1120, an examining device such as a cone-like structure mentioned above, may be configured to fit over the outlet of the lamp 104 and the wing-like flanges 25 on the lip retracting device 1138, as shown in FIG. 3a. The wing-like flanges 25 may be configured to interact with formations, such as slots 112 on the cone, to thereby provide a consistent and controlled gap between the lamp 104 and the teeth of the patient to be treated or examined.

In yet another embodiment, the wing-like members 25 may be adapted to be received by a dental light source so as to substantially fix the distance and orientation of the dental light source with respect to a patient, as shown in FIG. 3b, where the light source, for example, an illumination frame, includes at least one formation such as a slot, for engaging the wing-like members.

In FIG. 3b, the illumination frame 105 has a generally arcuate shape having a first end 501 and a second end 502. The back 460 of the illumination frame 105 is convex and the front 465 of the illumination frame 105 is concave. The illumination frame 105 may also serve as the spacer having formations. In other words, the spacer and formations, for example, slots, may both be present on the lamp housing, such as the illumination frame 105. The ends 501, 502 each has a slot 503, 504 open from the front 465 of the illumination frame 105 towards the back 460 of the illumination frame 105. Each slot, 503, 504, extends inwardly from its respective end, 501, 502, of the illumination frame 105.

In the illumination system with multiple light sources, the light sources may be collectively powered or individually powered. If individually powered, each of the individual light sources may be turned on or off separately, as desired. This is especially useful for a curing or imaging process, where only one or two teeth may be undergoing treatment or being examined at one time.

Multiple light sources may be arranged in a geometric arrangement. In one embodiment, they may be arranged in an arcuate form and may, for example, conform to the jaw of a patient, as shown in FIG. 3c. The illumination frame 105 has a front 465 and a back 460. The front 465 is concave and the back 460 is convex. The tube 115 is attached to the back 460 of the illumination frame 105. The tube 115 provides support for the illumination frame 105 and also acts as a conduit for wiring for the illumination frame 105. A plurality of light sources 235 may be arranged along the front 465 of the illumination frame 105. The light sources may be any light source as mentioned above. These light sources are merely exemplary and are not limited to those listed. The light sources 235 generate and direct light toward the patient's teeth for a whitening, imaging or a curing process. In a first embodiment, the light sources 235 emit light having substantially the same light spectrum. In a second embodiment, the light sources 235 emit light having different spectra. In one aspect, the light sources 235 may protrude from the surface of the front 460 of the illumination frame 105. In another aspect, the light sources 235 may be disposed substantially flush with the surface of the front 460 of the illumination frame 105. In a further aspect, the light sources are approximately equidistant from the various teeth toward which the light sources are directed. In yet another aspect, the frame may be rectangular.

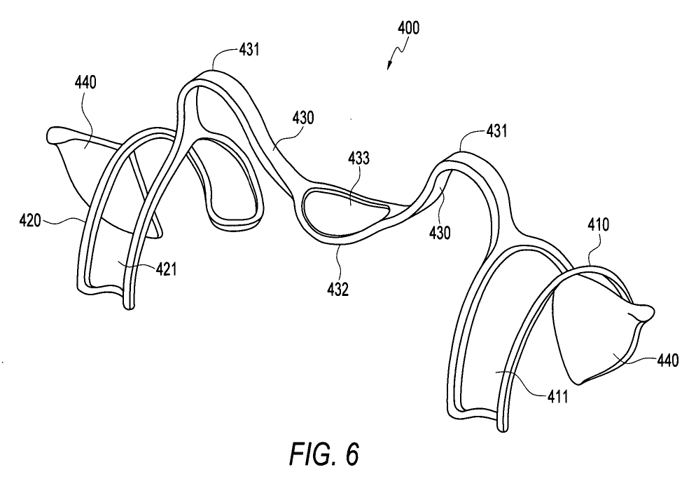

In some embodiments, the illumination frame 105 may be shaped and configured to mate with a reference device such as a lip retracting device worn by the patient, as shown in FIGS. 3 and 6, thereby providing a substantially precise alignment with the patient's mouth.

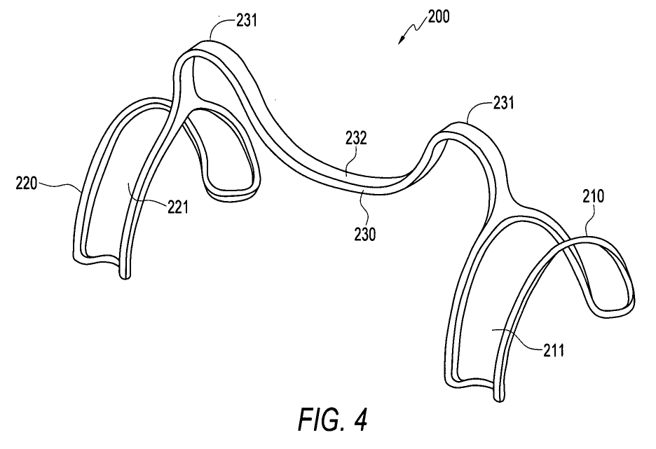

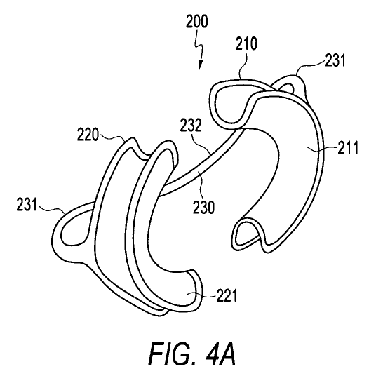

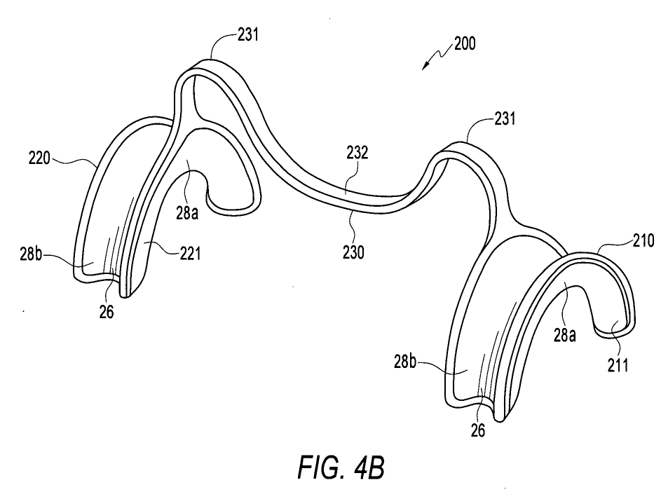

FIG. 4 shows an alternative embodiment of a retracting device in accordance with the present invention. The retracting device 200 may include a framework adapted to define the boundary and general shape of each of two lip channel retainers, 210, 220, and an elastomeric material contained within the frame work thereof, forming the bounded areas, 211, 221, respectively. Within the bounded areas, 211, 221, a suitable material, for example, such as the elastomeric material or foam mentioned above, may be mounted so as to form generally “U” shaped channels with the channel retainer frameworks, 210, 220. The elastomeric material may also be mounted in the manners described above, either mounted in a slightly stretched form or in a relaxed, non-stretched form. When used, the retracting device 200 draws back the lips, which retracts the cheeks, to expose the mouth so that a health care professional can more easily see the teeth and work on the teeth and/or mouth.

The two channel retainers, 210, 220, may retain the ends of the lips, for example, approximately where the upper and the lower lips intersect. More particularly, the two channel retainers or flanges, 210, 220, are adapted to cup the lips and to bias them open to expose the teeth for treatment and/or examination.

The elastomeric material is more comfortable for use on a patient and conformable to provide additional cushioning. It may also be adapted to follow the contour of the patient's lips better, as noted above.

At least one resilient member 230 connects the two lip channel retainers, 210, 220, and also serves as a biasing means as noted above. As further discussed below, when the retracting device, 200 is inserted into the mouth and the two channel retainers 210, 220, cup portions of the lips, the resilient member 230 provides a retractive force to retract the lips radially outwardly for examination and/or treatment. As shown, the resilient member 230 arches at points 231 to position the lip channel retainers, 210, and 220, in line with the lips of a patient. The resilient member 230 may also flex about a center portion 232 to properly align the retracting device 200 in the mouth.

The lip channel retainers, 210, 220, when mounted with a suitable material within the bounded areas, 211, 221, resemble curvilinear c-channels when the mounted material forms an arcuate race 26 and two channel side walls 28a and 28b, as shown in FIG. 4b. The channel side walls or surfaces may resemble a bell and include, for example, a maximum wall dimension at approximately the mid-point with two smaller tapered tips at the ends thereof. In one embodiment, the inside side wall 28b, which is intraoral, as further discussed below, may be slightly larger relative to the outside side wall 28a. However, the relative dimensions may be reversed or can be the same without deviating from the functionality of the lip retracting device 200.

The arcuate race may have a radius of curvature adapted to mimic the curvature of the side of the lips when the lips are in the open position. Because this curvature may vary depending on the size and age of the user or patient, the retracting device 200 may be implemented with varying radii of curvature to fit the varied shape of the particular user/patient. The arcuate race may also include an irregular curvature or two or more different radii of curvature. If implemented, the irregular curvature can vary the amount of retraction of the portion of the lip that is seated within the arcuate race to vary the amount of retraction between those portions of the lip.

The retracting device 200 may be made by injection molding a thermoplastic material such as polypropylene, polyethylene, polystyrene, or the like, as discussed above, especially by injection molding polypropylene and having a smooth and transparent finish. The retracting device may be made as a single piece or as separate components that may be subsequently assembled. The retracting device may also, owing to its framework structure, be formed by cutting a shape from a flat sheet of a suitable material, such as a thermoplastic, such as those mentioned above. The proper three-dimensional orientations for the various parts of the framework may be accomplished by heating and bending the plastic framework to achieve the desired shape. The shape may then be retained by cooling the framework.

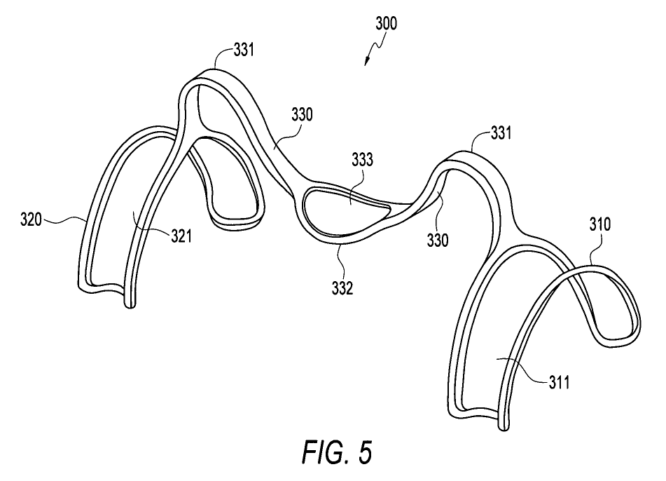

FIG. 5 shows a perspective view of another embodiment of a retracting device of the current invention. The retracting device 300 is substantially identical to the retracting device 200 of FIG. 4, with the only substantial difference being the addition of a tongue retaining framework 332 which defines the bounded area 333 in the middle of the resilient member 330. The bounded area 333 may be mounted with an appropriate material so as to form a cup for retaining the tongue in the back of the mouth when the retracting device 300 is in use on a patient, as discussed above. The material provides more comfort for use on a patient as the softness of the material may provide additional cushioning and the elasticity of the material allows contouring with the patient's tongue. Suitable materials may include various elastomers discussed above. When incorporated, the tongue retainer 332 and the resilient members 330 cooperate to block the tongue and limit the tongue to the back vicinity of the mouth, thus enabling access to the lingual portion or back of the teeth for examination and/or treatment. In short, the tongue retainer is configured to minimize interference by the tongue during treatment and/or examination by a health care professional, as noted above.

FIG. 6 shows a perspective view of another embodiment of a retracting device of the current invention. The retracting device 400 is substantially identical to the retracting device 300 of FIG. 5, with the only substantial difference being the addition of wing-like members 440 to the framework of lip channel retainers, 410, 420. The wing-like members 440 may be integrally molded or otherwise attached to the framework of the lip channel retainers 410, 420 by an adhesive or by heat sealing. The wing-like members 440 may be adapted to be received by a dental light source so as to substantially fix the distance and orientation of the dental light source with respect to a patient, as discussed above. The retracting device 400 may also include a tongue retainer 432, as described above. The tongue retainer 432 may also not be included in the present embodiment.

FIG. 7 shows a perspective view of a retracting device 500 which is substantially identical to the retracting device 200 of FIG. 4. The retracting device 500 is shown here with pads 540 attached to resilient member 530 between the center portion 532 and the arching portion 531. In other embodiments, the attachment may be made at other convenient portions of the retracting device. The pads 540 may be included to cushion the retracting device 500 against the inside of the patient's cheeks. The pads 540 may be molded out of any suitable material and may include either natural or synthetic rubber. Synthetic rubbers may be, for example, elastomeric materials and may include, but not limited to, various copolymers or block copolymers(Kratons®) available from Kraton Polymers such as styrene-butadiene rubber or styrene isoprene rubber; EPDM (ethylene propylene diene monomer) rubber; nitrile (acrylonitrile butadiene) rubber; latex rubber and the like. Foam materials may be closed cell foams or open cell foams, and may include, but is not limited to, a polyolefin foam such as a polyethylene foam, a polypropylene foam, and a polybutylene foam; a polystyrene foam; a polyurethane foam; any elastomeric foam made from any elastomeric or rubber material mentioned above; or any biodegradable or biocompostable polyesters such as those mentioned above.

The foam pads 540 may be molded in two halves connected along one edge, like a clam-shell, as shown in FIG. 8. The two halves may be fitted around the resilient member and then heat sealed together. Other methods of assembly that may achieve the same or similar results are also contemplated. For example, the pads 540 may incorporate a slot, channel, or ridge 541 for receiving at least a portion of the resilient member 530. For example, the ridges 109 may be so sized so that when the pad 540 are befitted over the resilient member 530, the pad may be adapted to slide relative to the resilient member to enable adjustment to the location of the pad 540 on the resilient member 530.

In other examples, the pads 540 may be made of polyethylene closed-cell foam so that they may be sterilized. Open cell foams may also be used if they are amenable to autoclaving, although non-sterilizable pads may also be used if the retracting device is designed for one-use, or if the pads are removable or replaceable. Also, the pads may have, for example, a smooth outside surface and smooth edges so that they may fit comfortably when in contact with the inside of a patient's mouth without unwanted irritation. In one embodiment, the pads 540 may be designed to be placed over the second to the last molar when the lip retracting device 10′ is inside a patient's mouth. In addition, they may be made with, for example, relatively high resiliency material so that they, for example, may return to their original shape after use.

In another embodiment, the pads 540 may be removable after each use, as noted above. Here, the pads may be attached using removable adhesive or the two halves may simply be mated over the resilient member using detents or the like. Any foam material that may be made to fit comfortably inside a patient's mouth may be used. The two-halves of the pad may also be made, for example, by heat set, to have a memory so that it may be forced open for installation onto a resilient member and be snapped shut when the opening force is removed. The two-halves may be integrally molded or attached along at least one side.

In a further embodiment, pads 540 may be permanently affixed and may not be sterilizable or autoclavable, as mentioned above, making the lip retracting device a one-patient use item.

The pads 540 may be attached with any suitable adhesive, such as an acrylic-based, a polyurethane-based, an epoxy-based, a polyamide-based, a cyanoacrylate-based adhesive, a styrene copolymer-based, a polyolefin-based or similar.

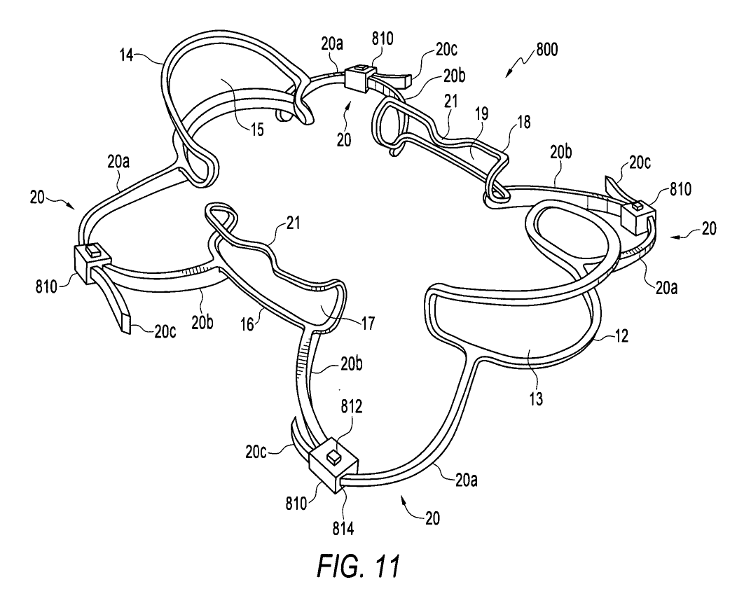

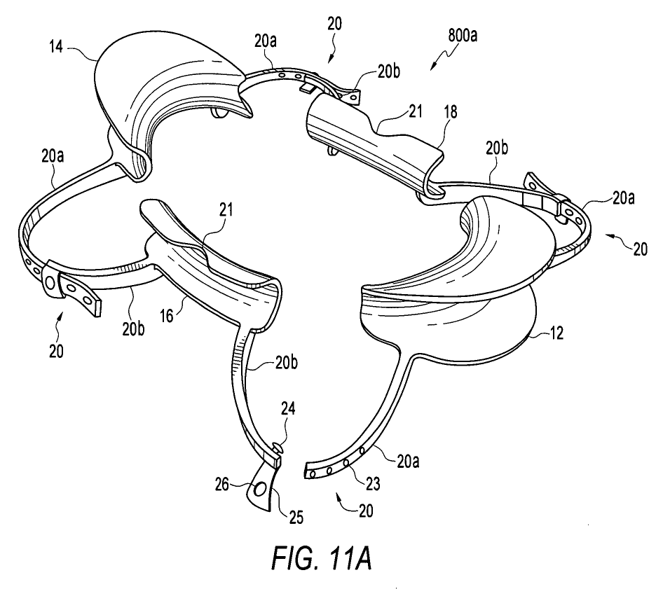

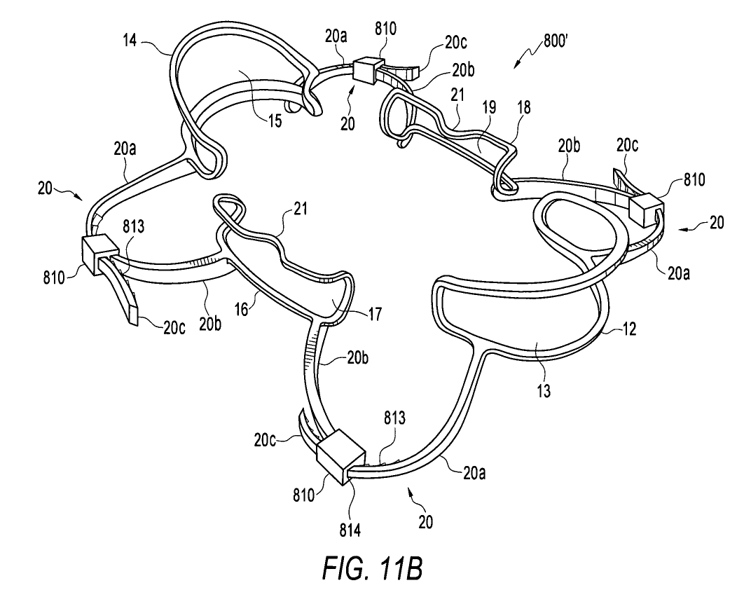

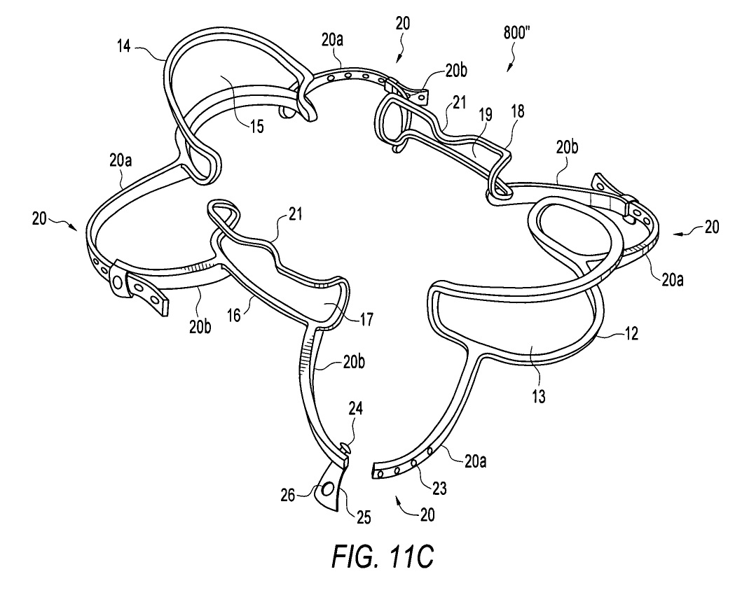

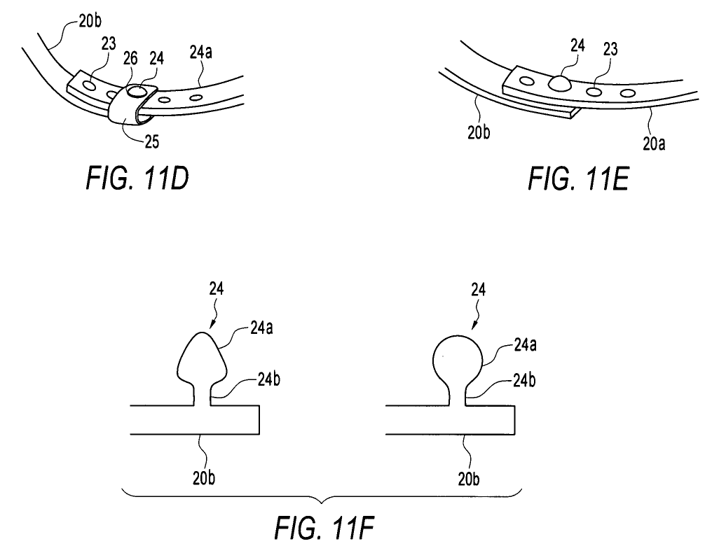

FIG. 11 shows a perspective view of another embodiment of a retracting device of the present invention. The retracting device 800 is substantially identical to the retracting device of any of the previously described device, for example that of FIG. 1, with the substantial difference that at least one of the resilient members 20 includes at least two portions, 20a and 20b, having inter-engaging formations.

In one embodiment, multiple adjustment features 810 are present on at least one portion. These adjustment features 810 are adapted to allow the length of the resilient members 20 to be varied. The adjustment features 810 may include a length control feature 812, which may include a spring-loaded latching mechanism. The length control feature 812 may be disposed on the adjustment feature 810, which may be mounted at approximately the end of portion 20b, however the position of 810 after engagement of 20a and 20b is complete may vary depending on the state of adjustment. The adjustment feature 810 may include a channel 814, adapted for slidably disposing a portion, 20c, towards the end of portion 20a of the resilient member 20. The channel 814 may serve to hold the portion 20c in place when in used, in cooperation with the length control feature 812. The length of the resilient members 20 may be varied by depressing the length control feature 812 and sliding the resilient member portion 20c through the channel 814 until the desired length is achieved. The length adjustment may then be fixed by releasing the length control feature 812, locking the resilient member portion 20c in place. The locking mechanism may have limited reversibility or substantial irreversibility. The adjustable mechanism enables the mass product of retracting devices of one size which may be later adjusted to fit each patient for better fit and more comfort.

In another embodiment, as shown in FIG. 11a, another adjustable retracting device 800a having limited reversibility or substantial irreversibility is shown. The lip retracting device includes four spaced apart channel retainers, 12, 14, 16, 18, also known as flanges, for retaining four corresponding portions of the lips for examination and/or treatment of the mouth or teeth. When used, the lip retracting device 800a acts like those exemplified in FIGS. 1a and 2a. The four channel retainers include two side channel retainers, 12, 14, for retaining the ends of the lips, approximately where the upper and the lower lips intersect, and two lip channel retainers, 16, 18, for retaining the mid-section of the upper and lower lips. More particularly, the four channel retainers or flanges, 12, 14, 16, 18, are adapted to cup the lips and bias them open to expose the teeth for treatment and/or examination, such as exemplified in FIGS. 1a and 1b, as noted above.

Similar to FIG. 1, a plurality of resilient members 20 are incorporated in the lip retracting device 800a to interconnect the four channel retainers, 12, 14, 16, 18, together and to also function as biasing means.

An optional tongue retainer 22 (not shown), similar to that shown in FIG. 2, may be approximately centrally positioned relative to the four channel retainers, 12, 14, 16, 18. The tongue retainer 22 may also be positioned asymmetrically about the two channel retainers 16 and 18. A trough 23 may be attached to the two channel retainers, 12, 14, by a pair of secondary resilient members 24, as is also shown in FIG. 2. When incorporated, the tongue retainer 22 and the secondary resilient members 24 cooperate to block the tongue and limit the tongue to the back vicinity of the mouth, thus enabling access to the lingual portion or the back portion of the teeth for examination and/or treatment.

In one embodiment, the tongue retainer 22 may include a framework having an elastomeric material disposed therein, as discussed above in relation to FIG. 2. In another embodiment, the tongue retainer may not be formed with a framework, while the channel retainers may each include a framework having an elastomeric material disposed therein.

In a further embodiment, an adjustable mechanism may be included in at least one of the resilient members, as discussed in FIG. 11 above, for adjusting the size of the retracting device. The locking mechanism may be reversible or irreversible.

In yet a further embodiment, as shown in FIG. 11a, a resilient member may include two separate portions, 20a and 20b. The portion 20a may include a plurality of holes 23 spaced at predetermined intervals to determine the adjustable length of the resilient member 20. The portion 20b may include a post 24. The end of the post 24 may be in the form of a bump or a bead, which may be of a round shape or a spear-shaped. The length of the resilient member 20 may be adjusted by inserting the post 24 into an appropriate hole 23 based on the desired length. This embodiment is shown in enlarged view in FIG. 11e, without a locking strap. The post may be formed integrally with the resilient member portion 20b or attached to the resilient member portion 20b by an adhesive or heat sealing. The permanency of the lock depends to a large extent on the shape and type of material of the post. The post may have a rounded or spear-shaped head 24a and a neck portion 24b, as exemplified in FIG. 11f. These constructions may allow a one-way locking mechanism without the possibility of removing the post 24 from the hole 23 once engaged, if the material used for the hole is not amenable to stretching and the material of the post 24 is not amenable to compression.

The embodiment as shown in FIG. 11a may include a locking strap 25 with locking feature 26. The locking feature 26 may include a hole, a button, or similar. After engaging post 24 with an appropriate hole 23, the locking strap 25 may then be folded over and the locking feature 26 may also engage the post 24 to lock it in place, making a permanent lock, as shown in FIG. 11d.

FIG. 11b shows another aspect of the embodiment of FIG. 11, where a different locking mechanism is used. In FIG. 11b, the length control feature 812 is absent. The channel 814 has prowls or teeth (not seen) for latching onto corresponding features 813 on the portion 20a. Again, this locking mechanism may or may not be permanent, depending of the shape and material of the prowls or teeth features 813 and 814. For example, even with the correct shapes for the teeth, if one of the materials for the teeth is amenable to stretching or compression, disengagement may be possible for repeated adjustment, for comfort and fit.

In another aspect, the adjustable locking mechanism shown in FIGS. 11a and 11e may be used in the embodiments of FIG. 11, in place of the locking mechanism shown in FIG. 11, as shown in FIG. 11c. As noted, the locking mechanism may or may not be permanent, depending of the shape and material of the post. For example, even with the shapes as shown in FIG. 11f, if one of the materials for the post or hole is amenable to stretching or compression, disengagement may be possible for repeated adjustment, for comfort and fit.

In other embodiments, where the adjustable mechanism is not present, any of the resilient members 20 may be formed as a single piece, integrally molded or attached by an adhesive or heat sealing to, for example, the outside side surface 28b and 44b of a pair of adjacent channel retainers 14, 16 or it may be formed in two halves separately and connected at the mid-portion, also integrally molded or attached by an adhesive or heat sealing to the outside side surface 28b and 44b of the adjacent channel retainers 14 and 16.

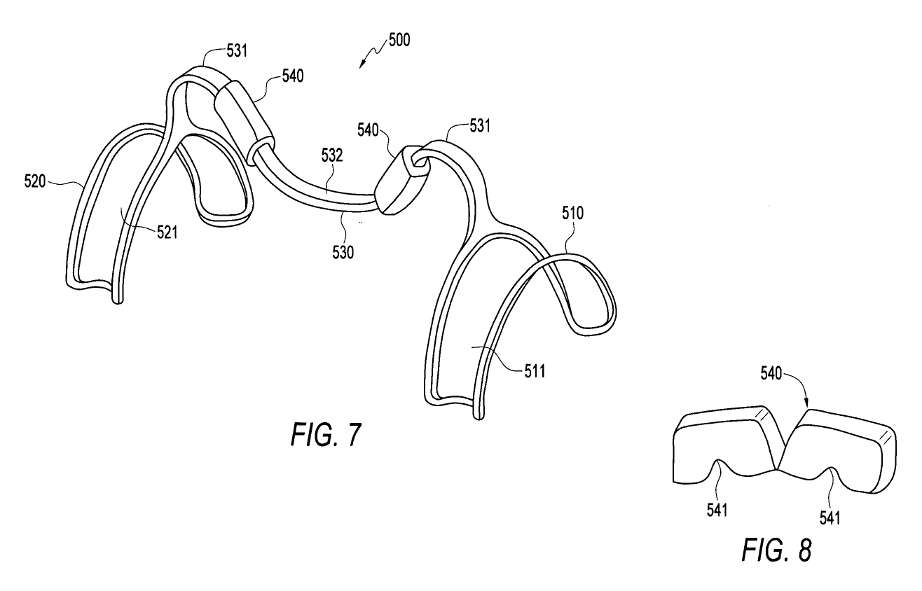

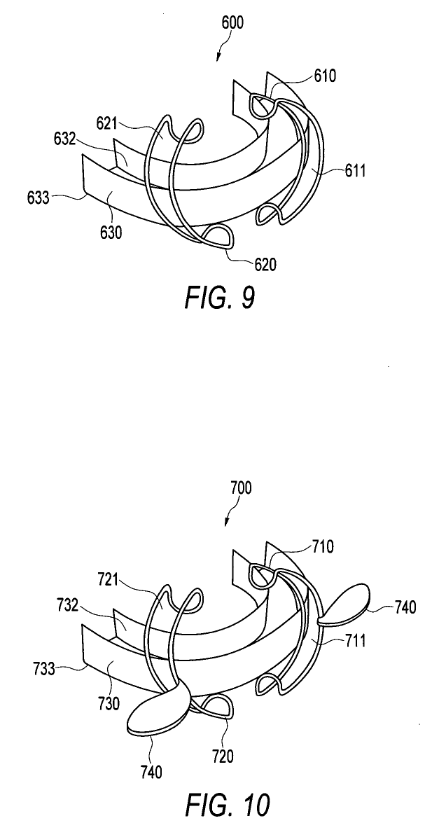

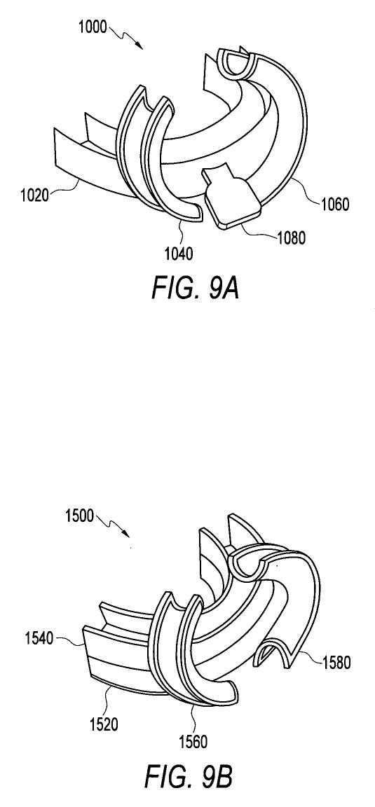

FIG. 9 shows a perspective view of a further embodiment of a retracting device of the present invention. The retracting device 600 includes two framework lip channel retainers, 610, 620, which define bounded areas, 611, 621, respectively. The bounded areas may be mounted with a suitable material, such as mentioned above, so as to form generally “U” shaped channels with the channel retainer frameworks, 610, 620. When used, the retracting device 600 works in a substantially similar manner to the retracting device of FIGS. 4-7. The retracting device 600 may include at least one non-inter-engaging formation. The non-inter-engaging formation may include a dental tray 630 adapted for positioning a dental composition against a subject's teeth. This dental tray 630 may also act in addition, as a resilient member for connecting the lip channel retainers, 610, 620. The dental tray 630 may also include channels, 632, 633, adapted to fit around both the upper and/or lower sets of teeth of a patient.

The dental tray 630 may be shaped or configured to accommodate a lower, or alternatively an upper, set of a patient's teeth, as noted. The additional u-shaped channel or channels, 632, 633, of the dental tray or trays 630 may be mounted substantially perpendicular to at least one of the lip retainer channel or channels, 610, 620. The lip retainer channels, 610, 620, are adapted to receive the lips of the dental patient and at least one of them supports the tray or trays 630 in substantially fixed spatial relation with respect to one another. At least one of the trays may function as a resilient member, or may be present in place of a resilient member.

The tray 630 may be formed by molding any suitable thermoplastic material, such as polypropylene, polyethylene, polystyrene, polycarbonate or others mentioned above for the retracting device. The tray 630 and the channels, 632, 633, may also be molded in accordance to a dental impression taken from a patient so as to provide a more exact fit to the patient's teeth.

According to one embodiment of the invention, as shown, for example, in FIG. 10, at least one inter-engaging formation, such as a wing-like member, may also be present for repeatably positioning a light source, or an imaging source to the subject undergoing dental procedure.

FIG. 10 shows a perspective view of another embodiment of a retracting device in accordance to the present invention. The retracting device 700 is substantially identical to the retracting device 600 of FIG. 9, with the only substantial difference being the addition of wing-like members 740 to the framework of lip channel retainers, 710, 720. As discussed above, the wing-like members 740 may be integrally molded or otherwise attached to the framework of the lip channel retainers, 710, 720. The wing-like members 740 may be adapted to be received by a dental light source so as to substantially fix the distance and orientation of the dental light source with respect to a patient.

In one embodiment, a pair of interface wings 740 may be coupled to the second and the third u-shaped channels, 710, 720, respectively. The interface wings 740 may be shaped and configured to be received into slots 1130 and 1132 in a light guide 1120, fitted to a lamp head or light source 1102, as shown in FIG. 3a, to align the light source 1102 with the teeth of a patient. To the extent that a patient's lips effectively serve to couple a lamp head and teeth of the patient in fixed relation to the lip retracting device 1138, the lip retracting device 1138 is maintained in a substantially fixed position with respect to a light source disposed within a lamp-head as shown, for example in FIG. 3a. This is described in greater detail below.

The interface wings or wing-like members, 740, like the wing-like members of the above described lip retracting devices, typically have some rigidity so that the interface wings 740 may form an effective interface when mated with the slots 1130, 1132 of the light guide 1120 or light source 1102, as seen in FIG. 3a. Similar to the embodiments described above, the interface wings 740 may in a first arrangement be formed of the same material as the channels, 710, 720, and/or 730. In a second arrangement, the interface wings 740 are made of a different material from the channels, 710, 720, and/or 730.

In use, the retracting device 700 is adapted to receive a dentistry composition, such as a dental whitening composition or a dental casting composition within the tooth receiving channels, 732, 733. The whitening composition may be light activatable or may be activatable in the dark. The teeth receiving channel may be transparent, translucent, or opaque, according to the desired application.

The lip retracting device may be flexible enough to be fitted to a patient and also rigid enough to support the whitening composition in place and to support the patient's lips. The lip retracting device may accordingly be made of any of the polymers mentioned above.

In an embodiment of the invention, the u-shaped channels may be made of the same material as the framework of the lip retainer channels, except that the lip channel retainers include frameworks having an elastomeric material therein. In a second embodiment of the invention, the first u-shaped channel is made of a different material from the second and third u-shaped channels again with the second and third u-shaped channels including frameworks having an elastomeric material therein. In a first exemplary arrangement, the first u-shaped channel may be made of rubber and the frameworks of the second and third u-shaped channels may be made of plastic. In a second exemplary arrangement, the first u-shaped channel may be made of a UV-permeable material while the second and third u-shaped channels may be made of a material that is substantially impermeable to UV light. In this embodiment, a light-activated composition may be used because the first u-shaped channel allows penetration of UV light to whiten the teeth while the second and third u-shaped channels provide some protection for the patient's soft tissues.

In another alternative embodiment, the first u-shaped channel may be made of a material including a whitening compound. In a first arrangement, the included whitening compound may be activated by a substance applied to the patient's teeth before the lip retracting device is applied to the patient. In a second arrangement, the included whitening compound may be activated by the saliva of the dental patient. In this embodiment, typically the exterior of the first u-shaped channel is covered with a layer of material that protects the patient's soft tissues. In a third arrangement, the included whitening compound is light-activated. In a fourth arrangement, the whitening composition may be activatable in the dark.

FIG. 9a shows a lip retracting device 1000 having a first u-shaped channel 1020 mutually coupled to the frameworks of a second u-shaped channel 1040 and third u-shaped channel 1060, which are substantially perpendicular to the first u-shaped channel 1020. A tab 1080 may be coupled to the first u-shaped channel 1020 between the second and third u-shaped channels, 1040, 1060. The tab 1080 may be useful for positioning the lip retracting device 1000 in the mouth of the dental patient. Later, after completion of the whitening or other dental process, the tab 1080 is useful for removing the lip retracting device 1000 from the mouth of the dental patient.

FIG. 9b is an alternative embodiment of the lip retracting device according to principles of the invention. The lip retracting device 1500 includes a lower jaw u-shaped channel 1520 and an upper jaw u-shaped channel 1540. The lower jaw u-shaped channel 1520 is shaped and configured to accommodate the lower set of the patient's teeth (not shown) while the upper jaw u-shaped channel 1540 is shaped and configured to accommodate the upper set of the patient teeth (not shown). Both the lower jaw u-shaped channel 1520 and the upper jaw u-shaped channel 1540 are adapted to receive a treatment composition, such as a whitening composition. The lip retracting device 1500 further includes a first u-shaped lip retracting device channel 1560 and a second u-shaped lip retracting device channel 1580 and they are mounted; substantially perpendicular to the lower jaw u-shaped channel 1520 and upper jaw u-shaped channel 1540. The lower jaw u-shaped channel 1520 and upper jaw u-shaped channel 1540 support a first u-shaped lip retracting device channel 1560 and a second u-shaped lip retracting device channel 1580 in substantially fixed spatial relation with respect to one another. The first and second u-shaped lip retracting device channels, 1560, 1580, are shaped and configured to receive the lips of the dental patient, both including frameworks and elastomeric materials therein, as discussed above.

In operation, the lower jaw u-shaped channel 1520 and the upper jaw u-shaped channel 1540 may each receive a treatment composition, such as a whitening composition. The lower jaw u-shaped channel 1520 and the upper jaw u-shaped channel 1540 may then be fitted over a patient's teeth so that the treatment composition such as a whitening composition is in contact with the patient's teeth. The patient's lips are received into the first and second u-shaped lip retracting device channels, 1560, 1580. Using the dental lip retracting device of the present embodiment, a dental process such as a whitening process may be performed on the lower teeth and the upper teeth at the same time effectively reducing the overall duration of the session.

In yet another embodiment of the present invention, a retracting device may include at least one non-inter-engaging formation. The non-inter-engaging formation may include a film holder. The device may also include at least one inter-engaging formation such as a wing-like member for repeatably positioning a light source or an imaging source to a subject undergoing dental procedure.

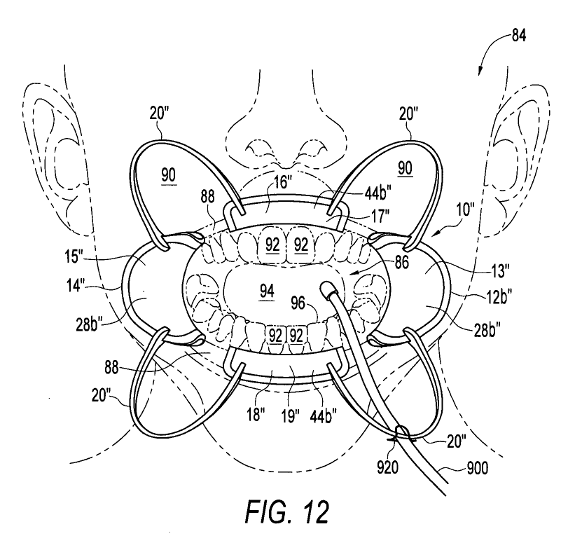

In one embodiment, the retracting device 10″ may include at least one formation adapted for mating with corresponding formations on an apparatus or tool adapted for aspiration, such as a suction tube and/or air/gas aspirator, or others, for positioning such apparatus in proximity to at least a portion of or inside a subject's mouth. The retracting device 10″ is shown in FIG. 12, where the formation is shown as a protrusion 20a, such as a post or a bump, disposed on one of the resilient members 20, adapted for mating with a clip 920 disposed on a suction tube 900, or vice versa. The formation may also be a depression, such as a dent, adapted for accepting a clip.

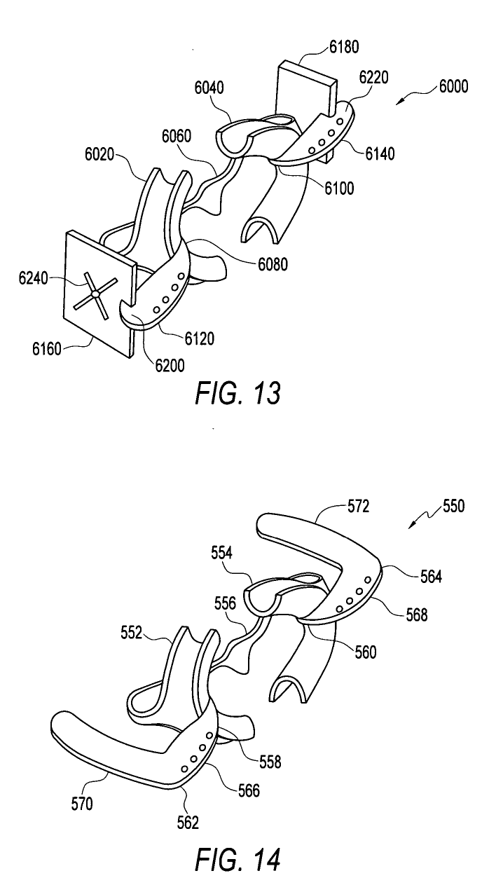

FIG. 13 shows a lip retracting device 6000 according to an alternative embodiment of the invention. The lip retracting device 6000 includes a first u-shaped channel 6020 and a second u-shaped channel 6040 to hold the lips of the patient whose teeth are to be imaged. A resilient or support member 6060 is mutually coupled to the u-shaped channels 6020, 6040 and may be adapted to support the u-shaped channels 6020, 6040 in substantially fixed spatial relation with respect to one another. A first end 6080 of the first interface wing 6120 is coupled to the first u-shaped channel 6020. A first end 6100 of the second interface wing 6140 is coupled to the second u-shaped channel 6040. The interface wings 6120, 6140 may be adapted to be received within the slots of a beam guide. A target 6160, 6180 is coupled to a second end 6200, 6220 of the first interface wing 6120 and second interface wing 6140 respectively. Each of the targets 6160, 6180 may have alignment markings 6240. In operation, the targets 6160, 6180 act as visual alignment mechanisms for the imaging head for side images of the patient's teeth.

FIG. 14 shows a lip retracting device 550 according to an alternative embodiment of the invention. The lip retracting device 550 has a first u-shaped channel 552 and a second u-shaped channel 554 to hold the lips of the patient whose teeth are to be imaged. A support member 556 is mutually coupled to the u-shaped channels, 552, 554, and may be adapted to support the u-shaped channels, 552, 554, in substantially fixed spatial relation with respect to one another. A first end 558 of the first interface wing 562 is coupled to the first u-shaped channel 552. A first end 560 of the second interface wing 564 is coupled to the second u-shaped channel 554.

Each of the interface wings, 562 or 564, includes a first portion, 566 or 568, located at the front of the lip retracting device 550 away from the patient. A second portion, 570 or 572, of each interface wing, 562, 564, extends outward and toward the back of the lip retracting device 550. In operation, the first portions, 566, 568, are located at the front of the patient's face while each second portion, 570 or 572, is located at a side of the patient's face. The interface wings, 562, 564, may be adapted to be received within the slots, 236, 238, of the beam guide 106. The first portions, 566, 568, enable the beam guide 106 to be aligned to the front of the patient. The second portions, 570, 572, of the interface wings, 562, 564, enable the beam guide 106 and imaging head 102 to be aligned on either side of the patient's head.

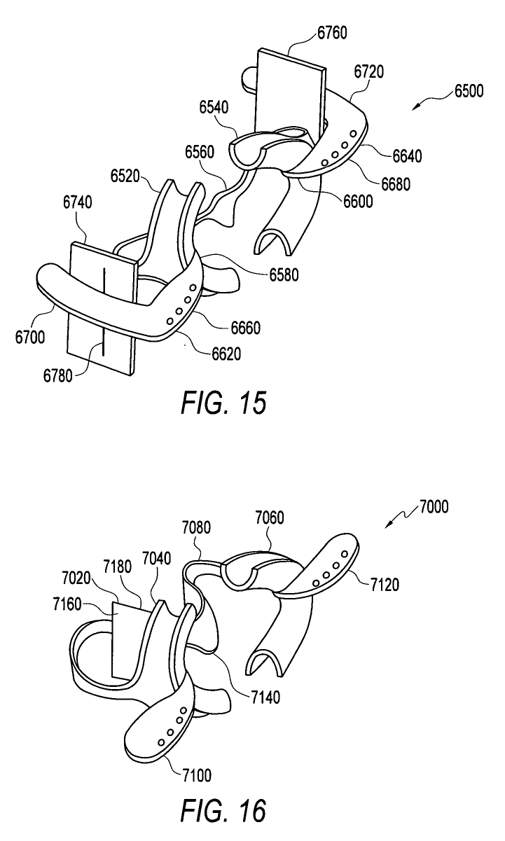

FIG. 15 shows a lip retracting device 6500 according to an alternative embodiment of the invention. The lip retracting device 6500 has a first u-shaped channel 6520 and a second u-shaped channel 6540 to hold the lips of the patient whose teeth are to be imaged. A resilient or support member 6560 is mutually coupled to the u-shaped channels, 6520, 6540, and may be adapted to support the u-shaped channels, 6520, 6540, in substantially fixed spatial relation with respect to one another.

A first end 6580 of the first interface wing 6620 is coupled to the first u-shaped channel 6520. A first end 6600 of the second interface wing 6640 is coupled to the second u-shaped channel 6540. Each interface wing, 6620 or 6640 includes a first portion, 6660 or 6680, located at the front of the lip retracting device 6500 away from the patient. A second portion, 5700 or 5720 of each interface wings, 6620 or 6640 extends outward and toward the back of the lip retracting device 6500. In operation, the first portions, 6660, 6680, are located at the front of the patient's face while each second portion, 6700 or 6720 is located at a side of the patient's face. The interface wings, 6620, 6640, may be adapted to be received within the slots of a beam guide. The first portions, 6660, 6680, enable the beam guide to be aligned to the front of the patient. The second portions, 6700, 6720, of the interface wings, 6620, 6640, enable the beam guide and imaging head to be aligned on either side of the patient's head.

A target, 6740, 6760, may be coupled to the second portions, 6700, 6720, of the first interface wing 6620 and second interface wing 6640 respectively. Each target, 6740 or 6760, may have visual alignment markings 6780. In operation, the targets, 6740, 6760, act as visual alignment mechanisms for the imaging head for side images of the patient.

FIG. 16 shows a lip retracting device 7000 according to one embodiment of the invention. The lip retracting device 7000 is also a fixturing device for maintaining the imaging head 1102 (as shown in FIG. 3a) in substantially fixed relation with respect to a target tooth. Further, the lip retracting device 7000 holds film inside the teeth of the patient in a manner in which the patient does not actively participate in the holding. In bite-wing images, for example, the patient bites down on a portion of the film holder in order to maintain the imaging film in a proper position. For people with poor muscle control, biting down for even a short period of time may be difficult. A film holder 7020 attached to the lip retracting device 7000 as described below may not require the active participation of the patient to hold the film in place.

As shown in the illustrated embodiment, the lip retracting device 7000 includes first 7040 and second 7060 semicircular “U”-shaped channels adapted to receive the lips of a dental patient, as described above with respect to FIG. 1a. A support member 7080 is mutually coupled to the “U”-shaped channels, 7040, 7060, and adapted to support the “U”-shaped channels, 7040, 7060, in substantially fixed spatial relation with respect to one another. According to one embodiment of the invention, a pair of interface wings, 7100, 7120, is coupled to the “U”-shaped channels, 7040, 7060, respectively. According to one embodiment of the invention, interface wings, 7100, 7120, are adapted to be received within slots 1130 and 1132 respectively (as shown in FIG. 3a).

The support member 7080 may be shaped and/or configured to support a film holder 7020. In one embodiment, the film holder 7020 may be an integral part of the lip retracting device 7000. In an alternative embodiment, the film holder 7020 may be separably coupled to the lip retracting device 7000. With the lip retracting device 7000 held in place by the patient's lips in the u-shaped channels, 7040, 7060, the film in the film holder 7020 may also be held in place. The lip retracting device 7000 is symmetric and accordingly the film holder could be located on either side of the central portion 7140 of the support member 7080. In an alternative embodiment of the invention, the support member 7080 may be configured to support two film holders 7020, one on either side of the central portion 7140 of the support member 7080. In this embodiment, the front 7160 of the film holder 7020 is x-ray penetrable while the back 7180 of the film holder 7020 blocks x-rays. In a first arrangement, the front 7160 of the film holder 7020 may be made of an x-ray penetrable material while the back 7180 of the film holder 7020 may be made of an x-ray blocking material. In a second arrangement, an x-ray blocking lining is inserted behind the film in the film holder 7020.

In a further embodiment of the invention, a voice alert system may be included in the lip retracting device itself. When the time period is concluded for any dental process, such as dental whitening, the voice alert system alerts the dental professional of the progress or completion of the procedure. A microelectronic component may be disposed on a portion of the retracting device. The microelectronic component may be self-contained so as to require no additional electronics, whether on a dental light source or other instrument. In one embodiment, the microelectronic component may include an electronic timing mechanism with a predetermined run time and a sound generator. The microelectronic component may also include an electromechanical interface, which may include, for example, a button, switch or other similar device. The electromechanical interface may serve to activate the electronic timing mechanism to begin the predetermined run time. The sound generator receives control signals from the timing mechanism and responsively produces linguistic audio signals in a human recognizable language corresponding to an elapsed or remaining time in a particular dental process. In another embodiment, the microelectronic component may also include a transmitting device which may include a radio transmitter, an infrared transmitter or other similar device. The transmitting device may be configured with a recorded voice message that when received by an appropriate receiver, may generate an audio message. The microelectronic component may also include a built-in power source, which may be removably or permanently connected to the microelectronic component. The power source may include a battery, a solar cell or other source of electrical energy.

In one embodiment, the microelectronic component may include an integrated circuit, which may include any or all of the above mentioned electronic components.

The microelectronic component may be mounted, for example, on the wing-like members of a lip retracting device as described above, or on any other portion. An electromechanical interface may be disposed, for example, in a portion of the wing-like members to effect the activation of the timing mechanism once the lip retracting device is mated with a dental light source, such as those described above. The electromechanical interface may also be disposed, in another example, on a portion of the lip retracting device such that it will be easily accessible by a dental healthcare professional for manual activation of the timing mechanism.

Although the exemplified embodiments of the invention have been described with some specificity, the description and drawings set forth herein are not intended to be limiting, and persons of ordinary skill in the art will understand that various modifications may be made to the embodiments discussed herein without departing from the scope of the invention, and all such changes and modifications are intended to be encompassed within the appended claims. Various changes to the lip retracting device may be made including manufacturing the dimensions differently, using different materials, making the lip retracting device opaque, semi-opaque, transparent, colored, having a textured finish, etc. For example, instead of making the lip retracting device by a single step injection molding, the lip retracting device can be made by welding the various components together, and using multiple molding steps. Also, the four channel retainers may be arranged such that two of the retainers cup two ends of the upper lip and two of the other retainers cup two ends of the lower lip. This alternative lip retracting device may also be incorporated with or without a tongue retainer. Accordingly, many alterations and modifications may be made by those having ordinary skill in the art without deviating from the spirit and scope of the invention.

1. A retracting device for retracting at least a portion of a user's mouth comprises:
at least two channel retainers, each comprising a framework having an elastomeric material disposed therein to form a race, an inside side wall, an outside side wall; and
at least one resilient member attached to the framework of two adjacent channel retainers.

2. The retracting device of claim 1 wherein at least one of said resilient members is integrally molded to the framework of the two adjacent channel retainers.

3. The retracting device of claim 1 further comprises at least one formation comprising wing-like members.

4. The retracting device of claim 3 wherein each of said wing-like members is adapted to fit into a formation in an apparatus comprising a an output port, a light guide, a light source, an imaging device or an examination device.

5. The retracting device of claim 1 wherein at least one of said resilient members comprises two portions, having a substantially rounded corss-section, connected by a pad.

6. The retracting device of claim 1 wherein the elastomeric material is selected from a group consisting of natural rubber, synthetic rubber, elastic foam or combinations thereof.

7. The retracting device of claim 1 wherein said retracting device comprises at least one pad attached to at least one resilient member.

8. The retracting device of claim 1 further comprising a tongue retainer, wherein the tongue retainer is attached to at least one of the channel retainers.

9. The retracting device of claim 1 wherein said device comprises at least four channel retainers or flanges, each comprising a race, an inside side wall, and an outside side wall, and four resilient members, each being integrally molded or attached to two outside side walls of two adjacent channel retainers.

10. The retracting device of claim 9 further comprises a tongue retainer attached to two of the channel retainers by at least one secondary resilient member.

11. The retracting device of claim 1 wherein at least one of said resilient members comprises at least one formation, said formation comprises a u-shaped channel adapted to accommodate at least one set of a subject's teeth.

12. The retracting device of claim 11 wherein said at least one u-shaped channel supports said at least one channel retainers in substantially fixed spatial relation with respect to one another.

13. The retracting device of claim 2 wherein said wing-like members are adapted to fit into at least one formation in a light guide, said light guide comprising at least one second formation adapted to be fitted into a light system.

14. The retracting device of claim 1 wherein at least one of said resilient members comprises at least one formation, said formation is adapted for accommodating an imaging film or sensor during imaging.

15. The retracting device of claim 14 wherein said formation comprises a film holder adapted to locate an imaging film, or an imaging sensor.

16. The retracting device of claim 15 wherein said imaging film, or imaging sensor is integrally formed with the holder.

17. The retracting device of claim 3 wherein at least one of said wing-like members comprises extended portions.

18. The retracting device of claim 3 wherein at least one of said wing-like members is adapted to be coupled to a target acting as a visual alignment mechanism for the imaging system.

19. The retracting device of claim 17 wherein at least one of said extended portions extends outward and toward the back of the retracting device.

20. The retracting device of claim 18 wherein said wing-like members are adapted to enable the imaging system to be aligned on either side of a subject's head.

21. The retracting device of claim 1 further comprises at least one formation comprising a tab for facilitating insertion and removal of the device.

22. The retracting device of claim 1 wherein at least one of said resilient members comprises at least two portions having corresponding formations that inter-engage when apposed.

23. The retracting device of claim 1 wherein at least one of said resilient members comprises an adjustable mechanism.

24. A retracting device comprises:
at least four channel retainers or flanges;
at least four resilient members, each of said resilient member is attached to two adjacent channel retainers; and
an adjustable mechanism for adjusting the length of the resilient member.

25. The retracting device of claim 24 wherein at least one of said resilient members comprises at least two portions having corresponding formations that inter-engage when apposed.

26. The retracting device of claim 24 wherein said adjustable mechanism comprises a permanent locking mechanism.

27. The retracting device of claim 24 wherein said device further comprises a tongue retainer attached to two of the channel retainers by at least one secondary resilient member.

28. The retracting device of claim 27 wherein said tongue retainer comprises a framework having a suitable elastomeric material disposed within the framework.

29. The retracting device of claim 24 further comprises at least one formation comprising wing-like members.

30. The retracting device of claim 29 wherein each of said wing-like members is adapted to fit into a formation in an apparatus comprising a an output port, a light guide, a light source, an imaging device or an examination device.

31. The retracting device of claim 27 wherein said tongue retainer comprises a framework having an elastomeric material disposed inside the framework.

32. The retracting device of claim 1 wherein said device further comprises a voice alert mechanism embedded in at least one portion of the retracting device.

33. The retracting device of claim 24 wherein said device further comprises a voice alert mechanism embedded in at least one portion of the retracting device.

34. The retracting device of claim 24 wherein at least one of said resilient members comprises at least one formation adapted for holding an apparatus adapted for aspiration.

35. A retracting device comprises:
at least two channel retainers, each retainer comprising a framework having disposed therein an elastomeric material defining a race, an inside side wall, an outside side wall; and
at least one formation comprising at least one u-shaped channel attached to the framework of at least one inside side wall of two adjacent channel retainers for repeatably positioning a subject's teeth with respect to a treatment composition.

36. The retracting device of claim 35 further comprises formations for positioning the retracting device to a dental system comprising a light system, a light guide, an imaging system or combinations thereof.

37. The retracting device of claim 35 wherein said u-shaped channel comprises a dental tray.

38. The retracting device of claim 35 wherein said at least one formation comprises at least two u-shaped channels.

39. A retracting device comprises:
at least two channel retainers, each retainer comprising a framework having disposed therein an elastomeric material defining a race, an inside side wall, an outside side wall;
at least one resilient member attached to the framework of at least one inside side wall of two adjacent channel retainers; and
at least one formation adapted for accommodating an imaging film or sensor during imaging.

40. The retracting device of claim 39 wherein said formation is coupled to a passively held portion adapted for providing alignment to at least one dental feature.

41. The retracting device of claim 40 further comprises an alignment formation coupled to an imaging device in a substantially fixed position with respect to the at least one dental feature.

42. The retracting device of claim 39 wherein at least one formation comprises a film holder coupled to a passively held portion.

43. The retracting device of claim 42 wherein said film holder is adapted to locate an imaging film, or an imaging sensor, for imaging the at least one dental feature.

44. The retracting device of claim 42 wherein said imaging film, or imaging sensor is integrally formed with the holder.

45. The retracting device of claim 39 wherein at least one formation comprises a wing-like member having extended portions.

46. The retracting device of claim 39 wherein at least one formation comprises wing-like members adapted to be coupled to a target acting as a visual alignment mechanism for the imaging system.

47. The retracting device of claim 45 wherein at least one of said extended portions extends outward and toward the back of the retracting device.

48. The retracting device of claim 45 wherein said wing-like members are adapted to enable the imaging system to be aligned on either side of a subject's head.

49. The retracting device of claim 1 further comprises at least one formation adapted for positioning an apparatus.

50. The retracting device of claim 49 wherein said at least one formation comprises a clip.

51. The retracting device of claim 49 wherein said apparatus comprises an air/gas aspirator, a suction tube or combinations thereof.

52. A retracting device comprises:
at least two channel retainers or flanges;
at least two resilient members, each of said resilient member is attached to two adjacent channel retainers; and
a tongue retainer;
wherein said tongue retainer comprises a framework having disposed therein an elastomeric material.

53. The retracting device of claim 52 wherein said elastomeric material is selected from a group consisting of natural rubber, synthetic rubber, elastic foam and combinations thereof.

54. The retracting device of claim 52 wherein said channel retainers comprises a framework having disposed therein an elastomeric material.

55. The retracting device of claim 52 wherein at least one of said resilient members comprises at least two portions having corresponding formations that inter-engage when apposed.

56. The retracting device of claim 1 wherein at least one of said resilient members comprises an adjustable mechanism.
